(19) United States
(12) Patent Application Publication
(10) Pub. No.: US 2010/0178320 A1
(43) Pub. Date: Jul. 15, 2010

(54) NEW MEDICAL PRODUCTS

(75) Inventor: Bengt Westrin, Stockholm (SE)

Correspondence Address: BIRCH STEWART KOLASCH & BIRCH, PO BOX 747, FALLS CHURCH, VA, 22040-0747, US

(73) Assignee: Lipopeptide AB, Stockholm (SE)

(21) Appl. No.: 12/666,116

(22) PCT Filed: Jun. 25, 2008
(86) PCT No.: PCT/GB2008/002192

§ 371 Date: Dec. 22, 2009

Related U.S. Application Data
(60) Provisional application No. 60/929,372, filed on Jun. 25, 2007.

(51) Int. Cl. A61K 9/70; A61K 38/02; A61K 38/17

(52) U.S. Cl. 424/443; 514/2; 514/12; 977/906; 977/755

(57) ABSTRACT

The present invention provides a wound care product comprising a wound care material and a polypeptide having wound care properties. In one embodiment, the wound care material comprises or consists of alginates, amorphous hydrogels, sheet hydrogels, hydrofibres, foams and mixtures thereof. In a further embodiment, the polypeptide having wound care properties is a cathelicidin, such as LL-37. The invention further provides methods of treatment of wounds using the products of the invention.

### FIELD OF INVENTION

The present invention relates to wound care products and uses of the same. In particular, the invention provides improved products for the treatment of chronic wounds.

### INTRODUCTION

Non-healing chronic wounds are a challenge to the patient, the health care professional, and the health care system. They significantly impair the quality of life for millions of people. Intensive treatment is required and imparts an enormous burden on society in terms of lost productivity and health care budget. Therefore, the study of healing chronic wounds is vitally important.

Wound healing is a dynamic pathway that optimally leads to restoration of tissue integrity and function. A chronic wound results when the normal reparative process is interrupted. By understanding the biology of wound healing, the physician can optimize the tissue environment in which the wound is present.

Healing pathways are set into motion at the moment of wounding. Wound healing is the result of the accumulation of processes, including coagulation, inflammation, ground substance and matrix synthesis, angiogenesis, fibroplasia, epithelialization, wound contraction, and remodelling. These complex overlapping processes are best organized into 3 phases of healing: the inflammatory phase, the proliferative phase, and the maturation phase.

Chronic Wounds

The above description of the wound healing process can be applied to both acute and chronic wounds. However, in the latter, the sequential process has been disrupted. When a wound proceeds through an orderly and timely reparative process and results in a sustained restoration of anatomic and functional integrity, it is termed an acute wound. Conversely, a chronic wound is one that has failed to proceed through the usual stepwise fashion. As a result, the healing process is prolonged and incomplete, with lack of restoration of integrity.

A chronic wound occurs when some factor causes the disruption of the normal, controlled inflammatory phase or the cellular proliferative phase. Many factors can contribute to poor wound healing. The most common include local causes such as wound infection; tissue hypoxia; repeated trauma; the presence of debris and necrotic tissue; and systemic causes such as diabetes mellitus, malnutrition, immunodeficiency, and the use of certain medications.

Wound infection is likely the most common reason for poor wound healing. All wounds are contaminated with bacteria. Whether a wound becomes infected is determined by the host's immune competence and the size of the bacterial inoculum. With normal host defenses and adequate debridement, a wound may bear a level of 100,000 (10⁵) microorganisms per gram of tissue and still heal successfully. Beyond this number, however, a wound may become infected.

Soft tissue cellulitis prolongs the inflammatory phase by inducing tissue proteases to degrade new granulation tissue and tissue growth factors and by delaying collagen deposition. Exudative fluid drawn from chronic wounds, in contrast to acute wounds, has elevated protease activity, diminished growth factor activity, and elevated levels of proinflammatory cytokines. Therefore, infection impedes healing by interfering with many steps in the normal progression from inflammation to proliferation to maturation of the wound.

Tissue perfusion may be impaired by arterial occlusion or vasoconstriction, hypotension, hypothermia, and peripheral venous congestion. Reduced wound oxygen tension can delay wound healing by slowing the production of collagen. Collagen fibril cross-linking begins to fail as tissue oxygen pressure falls below 40 mm Hg because oxygen is required for the hydroxylation of proline and lysine to synthesize mature collagen. Wound hypoxia also predisposes to bacterial infection because the leukocyte's oxidative phosphorylation bactericidal activities are severely impeded without normal tissue oxygen levels. These factors should be corrected as much as possible.

For example, hypoxia due to arterial occlusive disease can be improved by angioplasty or bypass grafting. The patient should be urged to cease using tobacco, which causes arterial vasoconstriction. A hypotensive or hypothermic patient should be properly resuscitated to improve cardiac function and blood volume as needed. Venous stasis is generally treated with compressive garments to improve vascular return. Anaemia is not detrimental to healing as long as the haematocrit value is greater than 15% and the patient is euvolemic. Because an adequate tissue oxygen tension directly correlates with the success of wound healing, optimizing oxygen tension is essential in all patients with any type of wound.

Devitalized tissue impairs healing because it provides a growth medium for bacteria, increasing the probability of infection. Dead tissue also exudes endotoxins that inhibit the migration of fibroblasts and keratinocytes into the wound. Foreign bodies such as suture material also fall into the category of debris when a wound is chronic in nature. The presence of a silk suture reduces the number of bacteria required to incite infection by a factor of 10,000. Therefore, debridement of all necrotic tissue and debris, whether performed by surgical means or with the use of enzymatic agents or wound dressings, is critical in achieving wound healing.

Underlying systemic disease in a patient with a wound can dramatically diminish the probability that the wound will heal in a timely fashion. Diabetes mellitus is a classic example. Wound healing is often delayed because of interruption of the inflammatory and proliferative phases. Neutrophils and macrophages cannot adequately keep the bacterial load of the wound controlled because their glycosylation is inhibitory to phagocytic function. Infection thus prolongs the inflammatory phase. When erythrocytes are affected by glycosylation (as measured by haemoglobin A1c levels), they become less pliable, leading to microvascular sludging and ischemia. Low tissue oxygen tension impairs cellular proliferation and collagen synthesis as previously described.

Malnutrition causes a decreased rate of fibroblastic proliferation and neovascularization and impairs both cellular and humoural immunity. A high rate of metabolic activity is present at the wound site, especially within new granulation tissue. If nutrients necessary for those activities are not provided, the health of the tissue is tenuous. Proteins and their amino acid building blocks, such as methionine, proline, glycine, and lysine, are essential for normal cell function and the repair of cutaneous wounds. Linolenic and linoleic acid must be supplied in the diet, which is why they are termed essential fatty acids.

Because they are critical constituents of the cell membrane and are the source of prostaglandins that mediate inflammation, deficiency of essential fatty acids causes impaired wound healing. Deficiency of vitamins C or K leads to scurvy and coagulopathy, respectively. Minerals, including calcium, iron, copper, zinc, and manganese, must be delivered to the wound milieu to act as cofactors for vital reactions in the synthesis of proteins needed in the healing process. If the diagnosis is impaired wound healing resulting from malnutrition, ensure that the patient receives adequate protein and energy (caloric) intake. Specific vitamin and mineral supplements may be required for rapid recovery of the necessary nutrients.

Finally, some medications prove to be detrimental to wound healing. Corticosteroids suppress inflammation at all levels, thereby blunting this phase of healing. Vitamin A reverses the negative effects of steroids and is indicated for topical and systemic application for all patients with chronic wounds who cannot discontinue corticosteroid therapy. Nonsteroidal anti-inflammatory agents such as aspirin and indomethacin interfere with the arachidonic acid cascade, impeding the elucidation of some of the healing scheme's primary mediators. Additionally, these act to inhibit the actions of platelets and platelet aggregation, thus disrupting the healing process from the first moment of wounding.

Treatment of Chronic Wounds

Traditional wound care products consist mainly of low technology gauze-based dressings such as woven and non-woven sponges, conforming bandages and non-adherent bandages. While effective in certain wound management environments, industry and commercial interest is focused on the wide range of new, advanced wound care products and treatments that are coming to market.

The advanced wound care segment encompasses a wide range of disparate technologies that fall into three main categories (see Ovington et al., 2007, Clinics in Dermatology 25:33-38):
Moist wound healing dressings (hydrogels, hydrocolloids, alginates, foams and transparent films);
(ii) Antimicrobial dressings which deliver substances such as silver to the wound;
(iii) Biological products such as skin substitutes, tissue-engineered products and growth factors.

In addition, a growing number of wound-healing devices such as negative pressure wound therapy (NPWT) are becoming more prominent. The sector also includes a variety of other treatments such as oxygen therapy, electrical stimulation; low level laser therapy (LLLT), therapeutic ultrasound and maggot therapy.

The US $4.1 billion global advanced wound care segment is the fastest growing area with double-digit growth of 10% per year. This growth is being driven by an ageing population, the rise in the incidence of diabetes worldwide and a steady advancement in technology and products that are more clinically efficient and cost effective than their conventional counterparts.

Hence, there exists an ongoing need for the development of improved medical products for the treatment and care of wounds.

### SUMMARY OF INVENTION

In a first aspect of the invention, there is provided a wound care product comprising a wound care material and a polypeptide having wound healing properties.

By “wound care product” we include products and devices which, when applied to a wound site, are able to aid (for example, accelerate) the wound healing process and/or to prevent infection of the wound. For example, the wound care product may be capable of enhancing epithelial regeneration and/or healing of wound epithelia and/or wound stroma. In one embodiment, the wound care product may be capable of enhancing the proliferation of epithelial and/or stromal cells through a non-lytic mechanism.

By “wound care material” we include substantially non-toxic materials suitable for use in wound care, including such wound care products as detailed below.

In one embodiment of the wound care products of the invention, the wound care material is capable of absorbing wound exudate.

The wound care material may be selected from the group consisting of alginates, amorphous hydrogels, sheet hydrogels, hydrofibres, foams and mixtures thereof.

Additional wound care materials, which are capable of absorbing wound exudate, include hydrocolloids, collagen-based materials, hyaluronic acid based materials, dextranomers, dextrinomer/cadexomer and oxidised regenerated cellulose.

For example, the wound care material may comprise or consist of an alginate. Wound care products comprising such wound care materials are typically provided in the form of a dry non-woven sheet (or ‘felt’), a freeze-dried sheet, a ribbon or a rope, and are particularly suitable for treating highly-exuding wounds.

Exemplary alginates available commercially include Suprasorb® (available from Sammons Preston, USA) and Kaltostat® (available from ConvaTec, UK).

Alternatively, the wound care material may comprise or consist of an amorphous hydrogel. Wound care products comprising such wound care materials are typically provided in the form of a viscous gel (e.g. in a tube or other applicator), and are particularly suitable for treating non-exuding wounds.

Suitable amorphous hydrogels may comprise one or more hydrogel-forming polymers selected from the group consisting of synthetic polymers, such as polyvinylalcohol, polyvinylpyrolidone, polyacrylic acid, polyethylene glycol, poloxamer block copolymers and the like; semi-synthetic polymers, such as cellulose ethers, including carboxymethylcellulose, hydroxyethylcellulose, hydroxypropylcellulose, methyl-cellulose, methylhydroxypropylcellulose and ethylhydroxyethylcellulose, and the like; natural gums, such as acacia, carragenan, chitosan, pectin, starch, xanthan gum and the like; and alginates.

Such hydrogel-forming polymers may be dissolved in an aqueous or non-aqueous solvent. Exemplary aqueous solvents include water, saline, buffers, water/propylene glycol and exemplary non-aqueous solvents include glycerol, propylene glycol and polyethylene glycol.

It is also advantageous to use block copolymers of the poloxamer type, i.e. polymers consisting of polyethylene glycol and polypropylene glycol blocks. Certain poloxamers dispersed in water are thermoreversible: at room temperature they are low viscous but exhibit a marked viscosity increase at elevated temperatures, resulting in a gel formation at body temperature. Thereby the contact time of a pharmaceutical formulation administered to the relatively warm wound cavity may be prolonged and thus the efficacy of an incorporated substance such as a polypeptide may be improved.

Exemplary hydrogels available commercially include Intrasite® (available from Smith & Nephew, UK) and Normigel® (available from Mölnlycke Health Care AB, Sweden).

Additionally, the wound care material may comprise or consist of a sheet hydrogel. As with amorphous hydrogels, such wound care materials are particularly suitable for treating non-exuding wounds.

Suitable sheet hydrogels may comprise one or more hydrogel-forming polymers selected from the group consisting of synthetic polymers, such as polyurethanes, polyvinylalcohol, polyvinylpyrolidone, polyacrylic acid, polyethylene glycol, poloxamer block copolymers and the like; semi-synthetic polymers, such as cellulose ethers, including carboxymethylcellulose, hydroxyethylcellulose, hydroxypropylcellulose, methylcellulose, methylhydroxypropylcellulose and ethylhydroxyethylcellulose, and the like; natural gums, such as acacia, carragenan, chitosan, pectin, starch, xanthan gum and the like; and alginates. Such hydrogel-forming polymers may be dissolved in an aqueous or non-aqueous solvent, as described above.

Exemplary sheet hydrogels available commercially include Elastogel® (available from Southwest Technologies Inc., USA) and Suprasorb® G (available from Sammons Preston, USA).

As a further alternative, the wound care material may comprise or consist of a hydrofibre. Wound care products comprising such wound care materials are typically provided in the form of a dry, non-woven sheet, freeze-dried sheet, or a ribbon or rope, and are particularly suitable for use with light-to-heavy exuding wounds or wounds with both dry and wet regions.

Suitable hydrofibres may comprise or consist of carbomethylcellulose, and include Aquacel® (available commercially from ConvaTec, UK).

As a further alternative, the wound care material may comprise or consist of a polyurethane foam, such as the Allevyn range of products (available from Smith&Nephew, United Kingdom)

A further key component of the wound care products of the present invention is a polypeptide having wound healing properties.

By “polypeptide having wound healing properties” we include polypeptides which are able to aid (for example, accelerate) the wound healing process and/or to prevent infection of the wound. For example, the wound care product may be capable of enhancing epithelial regeneration and/or healing of wound epithelia and/or wound stroma. In one embodiment, the polypeptide may be capable of enhancing the migration and/or proliferation of epithelial and/or stromal cells through a non-lytic mechanism.

It will be appreciated that such polypeptides having wound healing properties may have a primary or ancillary role in the function of the wound care products of the invention. By “polypeptide” we include pharmaceutically acceptable salts and derivatives thereof. For example, suitable pharmaceutically acceptable salts include those containing the counterions acetate, carbonate, phosphate, sulphate, trifluoroacetate and chloride. Suitable pharmaceutically acceptable derivatives include esters and amides.

In one embodiment, the polypeptide having wound healing properties is a cathelicidin, or a fragment, variant or fusion thereof which retains, at least in part, the wound healing activity of the parent cathelicidin.

For example, the cathelicidin may be selected from the group consisting of human cationic antimicrobial protein (hCAP18; see Accession Nos. NP_{—}004336 and AAH55089) and its C-terminal peptide LL-37, PR39, prophenin and indolicidin.

Human cathelicidin antimicrobial protein hCAP18, the only known cathelicidin in humans, consists of a conserved cathelin domain and a variable C-terminus, called LL-37 (Gudmundsson et al., 1996, Eur J Biochem 1238:325-32; Zanetti et al., 1995, FEBS Lett 374:1-5). Extracellular proteolytic processing of the holoprotein releases the LL-37 peptide, which has broad antimicrobial activity (Gudmundsson at al., 1995, Proc Natl Acad Sci USA 92:7085-9; Agerberth et al., 1995, Proc Natl Acad Sci USA 92:195-99) as well as effects on host cells, some of which are mediated by the G-protein-coupled receptor, formyl peptide receptor-like 1 (FPRL1) (Yang et al., 2000, J Exp Med 192:1069-74; Koczulla et al., 2003, J Clin Invest 111:1665-72). Human CAP18 is present in leucocytes (Cowland et al., 1995, FEBS Lett 368:173-76) and is expressed in skin and other epithelia where it is upregulated in association with inflammation (Cowland et al., 1995, FEBS Lett 368:173-76; Frohm et al., 1997, J Biol Chem 272:15258-63) and injury (Dorschner at al., 2001, J Invest Dermatol 117:91-97; Heilborn at al., 2003, J Invest Dermatol 120:379-89) consistent with a role in innate barrier protection.

In one embodiment, the polypeptide having wound healing properties is human LL-37, the amino acid sequence of which is shown below in SEQ ID NO:1:

[SEQ ID NO: 1]

LLGDFFRKSKEKIGKEFKRIVQRIKDFLRNLVPRTES

Thus, the polypeptide having wound healing properties may comprise or consist of the amino acid sequence of SEQ ID NO: 1.

The term ‘amino acid’ as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g. α,α-disubstituted amino acids, N-alkyl amino acids, etc.) and chemically derivatised amino acids (see below).

Preferably, however, the polypeptide, or fragment, variant, fusion or derivative thereof, comprises or consists of L-amino acids.

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for polypeptides used in the products of the present invention, as long as the desired functional property is retained by the polypeptide. For the peptides shown, each encoded amino acid residue, where appropriate, is represented by a single letter designation, corresponding to the trivial name of the conventional amino acid.

In an alternative embodiment, the polypeptide having wound healing properties is a biologically active fragment, variant, fusion or derivative of the amino acid sequence according to SEQ ID NO: 1.

By “biologically active” we mean that the fragment, variant, fusion or derivative retains, at least in part, the wound healing properties of the amino acid sequence according to SEQ ID NO: 1. For example, the fragment, variant, fusion or derivative may retain, at least in part, the ability of LL-37 to enhance epithelial regeneration and/or healing of wound epithelia and/or wound stroma. The retention of such wound healing properties may be determined using methods well known in the art (as disclosed in WO 2004/067025, which is incorporated herein by reference).

In one embodiment, the polypeptide having wound healing properties is a biologically active fragment of LL-37 comprising or consisting of at least 10 contiguous amino acids of SEQ ID NO: 1, for example at least 10, 11, 12, 13, 14, 15, 16, 17, 18, 19, 20, 21, 22, 23, 24, 25, 26, 27, 28, 29, 30, 31, 32, 33, 34, 35 or 36 contiguous amino acids of SEQ ID NO: 1. Thus, the fragment may comprise at least 10, 11, 12, 13, 14, 15, 16, 17, 18, 19, 20, 21, 22, 23, 24, 25, 26, 27, 28, 29, 30, 31, 32, 33, 34, 35 or 36 contiguous amino acids from the N-terminal (i.e. left) of SEQ ID NO: 1.

Thus, the polypeptide having wound healing properties may comprise or consist of a fragment of LL-37 selected from the group consisting of LL-36, LL-35, LL-34, LL-33, LL-32, LL-31, LL-30, LL-29, LL-28, LL-27, LL-26, LL-25, LL-24, LL-23, LL-22, LL-21 and LL-20 (as disclosed in WO 2004/067025, which is incorporated herein by reference).

In an alternative embodiment of the first aspect of the invention, the polypeptide having wound healing properties comprises or consists of a variant of the amino acid sequence according to SEQ ID NO: 1.

By ‘variant’ of the polypeptide we include insertions, deletions and substitutions, either conservative or non-conservative. For example, the variant polypeptide may be a non-naturally occurring variant.

It is particularly preferred that the variant has an amino acid sequence which has at least 50% identity with the amino acid sequence according to SEQ ID NO: 1 or a fragment thereof, for example at least 55%, 60%, 65%, 70%, 75%, 80%, 90%, 95%, 96%, 97%, 98% or at least 99% identity.

The percent sequence identity between two polypeptides may be determined using suitable computer programs, for example the GAP program of the University of Wisconsin Genetic Computing Group, and it will be appreciated that percent identity is calculated in relation to polypeptides whose sequences have been aligned optimally.

The alignment may alternatively be carried out using the Clustal W program (as described in Thompson et al., 1994, Nuc. Acid Res. 22:4673-4680, the relevant disclosures in which document are hereby incorporated by reference).

The parameters used may be as follows:
Fast pairwise alignment parameters: K-tuple (word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

Variants may be made using the methods of protein engineering and site-directed mutagenesis well known in the art (see example, see Molecular Cloning: a Laboratory Manual, 3rd edition, Sambrook & Russell, 2001, Cold Spring Harbor Laboratory Press, the relevant disclosures in which document are hereby incorporated by reference).

In a further alternative embodiment of the first aspect of the invention, the product comprises or consists of a fusion protein of which part corresponds to the amino acid sequence of LL-37 or a biologically active fragment or variant thereof.

By ‘fusion’ of a protein or polypeptide we include a polypeptide fused to any other polypeptide. For example, the said polypeptide may be fused to a polypeptide such as glutathione-S-transferase (GST) or protein A in order to facilitate purification of said polypeptide. Examples of such fusions are well known to those skilled in the art. Similarly, the said polypeptide may be fused to an oligo-histidine tag such as His6 or to an epitope recognised by an antibody such as the well-known Myc tag epitope. Fusions to any fragment, variant or derivative of said polypeptide are also included in the scope of the invention. It will be appreciated that fusions (or variants or derivatives thereof) which retain desirable properties, namely anticancer activity are preferred. It is also particularly preferred if the fusions are ones which are suitable for use in the methods described herein.

For example, the fusion may comprise a further portion which confers a desirable feature on the said polypeptide of the invention; for example, the portion may be useful in detecting or isolating the polypeptide, or promoting cellular uptake of the polypeptide. The portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the polypeptide, as known to those skilled in the art.

It will be appreciated by skilled persons that the polypeptide, or fragment, variant, fusion or derivative thereof, may comprise one or more amino acids that are modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. Thus, by ‘polypeptide’ we include peptidomimetic compounds which exhibit wound healing activity. The term ‘peptidomimetic’ refers to a compound that mimics the conformation and desirable features of a particular polypeptide as a therapeutic agent.

For example, the polypeptides described herein include not only molecules in which amino acid residues are joined by peptide (—CO—NH—) linkages but also molecules in which the peptide bond is reversed. Such retro-inverso peptidomimetics may be made using methods known in the art, for example such as those described in Meziere et al. (1997) J. Immunol. 159, 3230-3237, the relevant disclosures in which document are hereby incorporated by reference. This approach involves making pseudopeptides containing changes involving the backbone, and not the orientation of side chains. Retro-inverse peptides, which contain NH—CO bonds instead of CO—NH peptide bonds, are much more resistant to proteolysis. Alternatively, the polypeptide of the invention may be a peptidomimetic compound wherein one or more of the amino acid residues are linked by a -γ(CH₂NH)— bond in place of the conventional amide linkage.

In a further alternative, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond.

It will be appreciated that the polypeptide may conveniently be blocked at its N- or C-terminus so as to help reduce susceptibility to exoproteolytic digestion, e.g. by amidation.

A variety of uncoded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell et al., 1983, Biochem. Biophys. Res. Comm. 111:166, the relevant disclosures in which documents are hereby incorporated by reference.

A common theme among many of the synthetic strategies has been the introduction of some cyclic moiety into a peptide-based framework. The cyclic moiety restricts the conformational space of the peptide structure and this frequently results in an increased affinity of the peptide for a particular biological receptor. An added advantage of this strategy is that the introduction of a cyclic moiety into a peptide may also result in the peptide having a diminished sensitivity to cellular peptidases.

Thus, preferred polypeptides comprise terminal cysteine amino acids. Such a polypeptide may exist in a heterodetic cyclic form by disulphide bond formation of the mercaptide groups in the terminal cysteine amino acids or in a homodetic form by amide peptide bond formation between the terminal amino acids. As indicated above, cyclising small peptides through disulphide or amide bonds between the N- and C-terminus cysteines may circumvent problems of affinity and half-life sometime observed with linear peptides, by decreasing proteolysis and also increasing the rigidity of the structure, which may yield higher affinity compounds. Polypeptides cyclised by disulphide bonds have free amino and carboxy-termini which still may be susceptible to proteolytic degradation, while peptides cyclised by formation of an amide bond between the N-terminal amine and C-terminal carboxyl and hence no longer contain free amino or carboxy termini. Thus, the peptides of the present invention can be linked either by a C—N linkage or a disulphide linkage.

The present invention is not limited in any way by the method of cyclisation of peptides, but encompasses peptides whose cyclic structure may be achieved by any suitable method of synthesis. Thus, heterodetic linkages may include, but are not limited to formation via disulphide, alkylene or sulphide bridges. Methods of synthesis of cyclic homodetic peptides and cyclic heterodetic peptides, including disulphide, sulphide and alkylene bridges, are disclosed in U.S. Pat. No. 5,643,872. Other examples of cyclisation methods are discussed and disclosed in U.S. Pat. No. 6,008,058, the relevant disclosures in which documents are hereby incorporated by reference.

A further approach to the synthesis of cyclic stabilised peptidomimetic compounds is ring-closing metathesis (RCM). This method involves steps of synthesising a peptide precursor and contacting it with an RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Another approach, disclosed by D. H. Rich in Protease Inhibitors, Barrett and Selveson, eds., Elsevier (1986; the relevant disclosures in which document are hereby incorporated by reference), has been to design peptide mimics through the application of the transition state analogue concept in enzyme inhibitor design. For example, it is known that the secondary alcohol of staline mimics the tetrahedral transition state of the scissile amide bond of the pepsin substrate.

In summary, terminal modifications are useful, as is well known, to reduce susceptibility by proteinase digestion and therefore to prolong the half-life of the peptides in solutions, particularly in biological fluids where proteases may be present. Polypeptide cyclisation is also a useful modification and is preferred because of the stable structures formed by cyclisation and in view of the biological activities observed for cyclic peptides.

Thus, in one embodiment the polypeptide, or fragment, variant, fusion or derivative thereof, is cyclic. However, in a preferred embodiment, the polypeptide, or fragment, variant, fusion or derivative thereof, is linear.

Methods for the production of polypeptides, or fragment, variant, fusion or derivative thereof, for use in the first aspect of the invention are well known in the art. Conveniently, the polypeptide, or fragment, variant, fusion or derivative thereof, is or comprises a recombinant polypeptide.

Thus, a nucleic acid molecule (or polynucleotide) encoding the polypeptide, or fragment, variant, fusion or derivative thereof, may be expressed in a suitable host and the polypeptide obtained therefrom. Suitable methods for the production of such recombinant polypeptides are well known in the art (for example, see Sambrook & Russell, 2000, Molecular Cloning, A Laboratory Manual, Third Edition, Cold Spring Harbor, N.Y., the relevant disclosures in which document are hereby incorporated by reference).

In brief, expression vectors may be constructed comprising a nucleic acid molecule which is capable, in an appropriate host, of expressing the polypeptide encoded by the nucleic acid molecule.

Polypeptides can also be produced in vitro using a commercially available in vitro translation system, such as rabbit reticulocyte lysate or wheatgerm lysate (available from Promega). Preferably, the translation system is rabbit reticulocyte lysate. Conveniently, the translation system may be coupled to a transcription system, such as the TNT transcription-translation system (Promega). This system has the advantage of producing suitable mRNA transcript from an encoding DNA polynucleotide in the same reaction as the translation.

The present invention also includes products comprising pharmaceutically acceptable acid or base addition salts of the above described wound healing polypeptides. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically acceptable base addition salts may also be used to produce pharmaceutically acceptable salt forms of the polypeptides. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

Thus, in the products of the present invention LL-37 or fragment thereof may be used in the form of an acetate salt.

It will be appreciated by persons skilled in the art that the polypeptide having wound healing properties may be formulated initially in any suitable medium/buffer, such as PBS or ethanol, before being admixed with or applied to the wound care material.

In one embodiment of the wound care products of the invention, the weight ratio of the wound care material to the polypeptide having wound healing properties is equal to or greater than 10:1, for example equal to or greater than 30:1, 100:1, 1000:1, 2000:1, 5000:1, 10000:1 or greater than 50000:1. For example, the weight ratio of the wound care material to the polypeptide having wound healing properties is equal to or greater than 10000:1.

Exemplary wound care products of the invention may comprise or consist of the following component combinations:
(a) the wound care material comprises or consists of polyurethane foam and the polypeptide having wound healing properties is LL-37;
(b) the wound care material comprises or consists of a hydrocolloid dressing and the polypeptide having wound healing properties is LL-37;
(c) the wound care material comprises or consists of an alginate felt a methylcellulose gel and the polypeptide having wound healing properties is LL-37:
(d) the wound care material, comprises or consists of a methylcellulose gel and the polypeptide having wound healing properties is LL-37; and
(e) the wound care material comprises or consists of an acacia hydrogel (Arabic gum) and the polypeptide having wound healing properties is LL-37.

In one particular embodiment, the wound care product does not comprise a complex of LL-37 (or a fragment thereof) with a bilayer-forming lipid (such as a galactolipid).

In further embodiment of the first aspect of the invention, the wound care product further comprises an antimicrobial polypeptide, for example selected from group consisting of defensins, gramicidin S, magainin, cecropin, histatin, hyphancin, cinnamycin, burforin 1, parasin 1 and protamines, and fragments, variants and fusion thereof which retain, at least in part, the antimicrobial activity of the parent protein.

In a further embodiment of the wound care products of the invention, the polypeptide having wound healing properties (such as LL-37) is released slowly in use. For example, less than 50% of the polypeptide having wound healing properties contained in the wound care product may be released within the first 24 hours of use, for example less than 40%, 30%, 20%, 10% or 5%. Release rates may be measured using the methods described in the Examples below.

The wound care products of the present invention may take a number of different forms, depending on the constituent materials used and the intended purpose of the product. Typically, however, the product is provided in the form of a dressing. For example, the product may take the form of a polyurethane foam, dry non-woven sheets, freeze-dried sheets, solid gel sheets, ribbons, ropes and viscous gels.

Prior to use, the wound care product should be sterile and packaged in a microorganism-impermeable container. For example, the wound care product may be stored in a tube or other suitable sterile applicator.

Sterility may be achieved using techniques well known in the art, such as aseptic manufacturing and/or final (i.e. post-production) sterilisation by irradiation.

Persons skilled in the art will appreciate that the wound care products of the invention may be suitable for maintaining a moist wound environment. Thus, the product may comprise wound care materials capable of either adding moisture to a wound or removing moisture from a wound.

Advantageously, the wound care product is capable of preventing, abolishing, reducing or otherwise diminishing microbial growth in a wound environment.

It will be appreciated that the wound care products of the invention may be sized and shaped to fit wounds at various sites on the body. For example, the wound care products may be shaped to provide a wound dressing surface which is substantially planar (i.e. flat), concave, convex, etc.

Thus, the wound care products may be substantially planar (i.e. flat) with a thickness (average or maximum) equal to or less than 20 mm, for example equal to or less than 10 mm, 8 mm, 6 mm, 5 mm, 4 mm, 3 mm, 2 mm or 1 mm.

In one particular embodiment of the first aspect of the invention, the wound care product comprises or consists of a layer of wound care material to which is attached, on the wound-facing side, a film containing the polypeptide having wound healing properties.

For example, the wound care material layer may comprise or consist of a polyurethane foam dressing, a hydrocolloid sheet dressing, a hydrogel sheet or a non-aqueous gel sheet. Advantageously, the wound care material layer is capable of absorbing wound exudate.

The film component of the exemplary wound care product, containing the polypeptide having wound healing properties, may be attached directly to a surface of the wound care material layer. Alternatively, the film may be attached indirectly via one or more intervening layers or films (see below).

Typically, the film will comprise a film forming material and the polypeptide having wound healing properties. The film may also comprise additional components, such as a plasticizer and colourants.

Suitable film forming materials are well known in the art, such as synthetic polymers, starches and polysaccharides. For example, the film may be formed from an aqueous polymer matrix, cellulose derivatives, acrylate copolymers, gums, polysaccharides and polylactic acid polymers.

Preferably, the film is water-soluble.

The film composition may be chosen to provide a specific, controllable dissolution rate.

For example, the film may have dissolution time (measured either on the wound or in water) of less than 1 hour, for example less than 30 minutes, 20 minutes, 10 minutes or 5 minutes. Dissolution time may be controlled by the selection of appropriate film forming material; for example, polysaccharides may provide fast dissolution (<10 seconds), hydroxypropyl methyl cellulose may provide a medium dissolution speed (about 30 seconds), while corn starch may provide slower dissolution (>2 minutes).

Typically, the film is equal to or less than 1 mm thick, for example equal to or less than 0.8 mm, 0.6 mm, 0.4 mm, 0.2 mm, 0.1 mm or 0.05 mm.

The polypeptide having wound healing properties may be evenly distributed within the film (for example, the polypeptide may be added to a film forming material, such as an aqueous polymer matrix, prior to formation of the film layer).

The film component of the exemplary wound care product may cover all or just part of the wound-facing side of the wound care material layer. Thus, the film may cover at least 30% of the surface area of one side of the wound care material layer, for example at least 50%, 60%, 70%, 80%, 90% or 100% of the surface area.

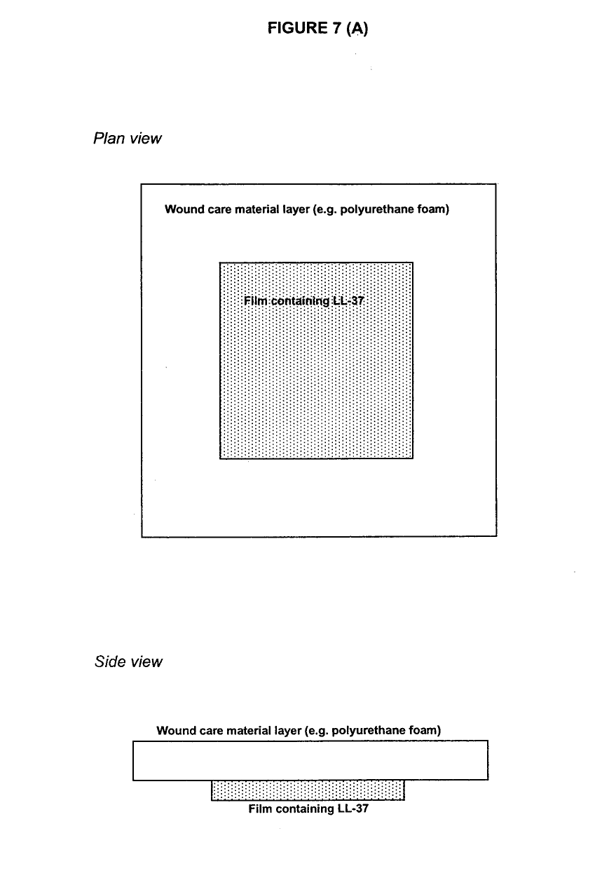

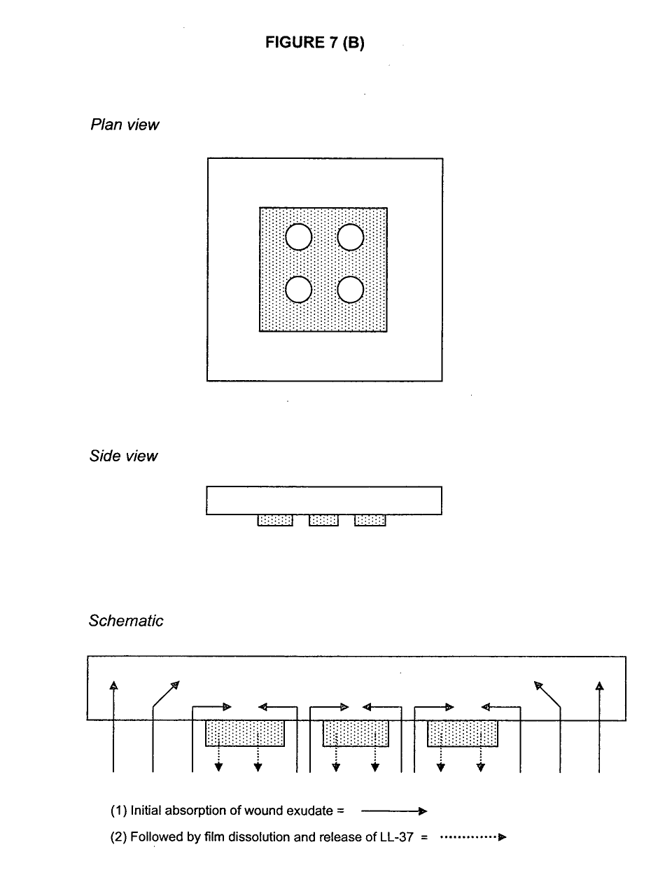

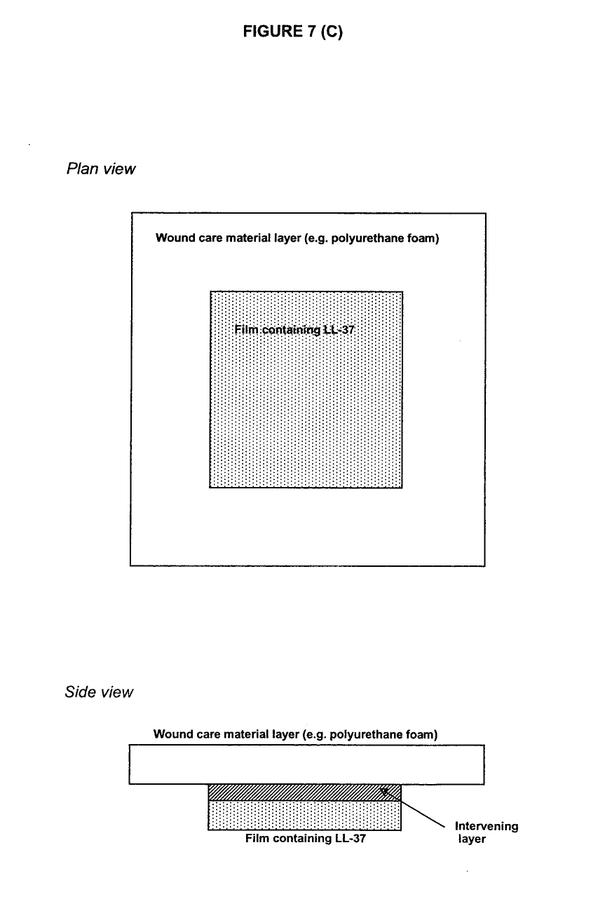

Conveniently, the film covers a central portion of the wound care material layer surrounded by an exposed peripheral region of the wound care material layer (see FIG. 7).

In one embodiment, the film is perforated. Perforations in films are particularly useful for exuding wounds, since they can reduce or prevent backwash of the polypeptide having wound healing properties onto the wound care material. Thus, the perforations allow the initial wound exudate to absorb onto the wound care material, after which time LL-37 can slowly be realised from the soluble film into the wound site.

The extent of perforation and size of the perforations may be optimised for wound healing performance. For example, the perforations may account for at least 10%, 20%, 30%, 40%, 50% or more of the surface area of the film. The individual perforations may have an average size of at least 0.1 mm², for example at least 0.2 mm², 0.5 mm², 1 mm², 2 mm², 5 mm² or more.

In one embodiment, the film containing the polypeptide having wound healing properties is attached indirectly to the wound care material layer, via an intervening layer. The intervening layer preferably has a lower dissolution rate than the film containing the polypeptide having wound healing properties. For example, the intervening layer may have dissolution time (measured either on the wound or in water) of more than 5 minutes, for example more than 10 minutes, 20 minutes, 30 minutes or 60 minutes.

It will be appreciated that the intervening layer may also be perforated (like the film). Optionally, the perforations in the intervening layer coincide (i.e. align) with the perforations in the film. Alternatively, however, the perforations in the intervening layer may be offset from the perforations in the film.

Exemplary embodiments of the above wound care product designs include the following:
(a) A wound care product capable of absorbing wound exudate comprising a polyurethane foam dressing to which is attached, on the side to be contacted with the wound, a non-perforated water-soluble film containing LL-37;
(b) A wound care product capable of absorbing wound exudate comprising a polyurethane foam dressing to which is attached, on the side to be contacted with the wound, a perforated water-soluble film containing LL-37;
(c) The wound care product of (a) or (b) wherein the film is attached indirectly to the polyurethane foam dressing via a non-perforated water-soluble intervening layer (having lower-dissolution rate than the film); and
(d) The wound care product of (a) or (b) wherein the film is attached indirectly to the polyurethane foam dressing via a perforated water-soluble intervening layer (having lower water-solubility than the film).

Examples of such wound care product designs are shown in FIG. 7.

A second aspect of the invention provides the use of a wound care product as detailed above in the treatment of wounds. Such products are particularly suited to the treatment of chronic wounds, for example venous ulcers, diabetic ulcers and pressure ulcers.

Typically, the wound care product is applied directly to the surface of the wound. Optionally, a secondary conventional dressing may be applied over the top of the wound care product. Furthermore, in some cases, a permeable anti-adherence dressing may be applied between the wound and the wound care product.

It will be appreciated the products of the invention should be replaced on the wound at regular intervals, to aid the healing process and to prevent infection.

A third aspect of the invention provides a method for treating a wound comprising contacting the wound with a wound care product as detailed above.

A fourth aspect of the invention provides a method of producing a wound care product comprising combining a wound care material and a polypeptide having wound healing properties. The method may comprise admixing the wound care material and the polypeptide such that the polypeptide is dispersed through the wound care material; this may be done either before or during preparation of the wound care material. Alternatively, the polypeptide having wound healing properties can be applied to an exposed surface of the wound care material, after such wound care material has been prepared. In a further alternative, a film comprising the polypeptide having wound healing properties is attached or applied to the wound care material.

For example, in the case of wound care products comprising an alginate wound care material, the polypeptide having wound healing properties may be added before, during or after the manufacture of the wound care material. Thus, the wound healing polypeptide may be added before the fibre spinning (e.g. wet spinning) process in the case of non-woven sheets, or before the freeze-drying process in the case of freeze-dried sheets. Alternatively, an aqueous or non-aqueous solution of the polypeptide having wound healing properties can be applied after the manufacture of the wound care material, followed by a drying step (which may optionally be freeze-drying or vacuum drying).

In the case of wound care products based on a hydrofibre wound care material and comprising a polypeptide having wound care properties, these may be manufactured in a similar way as described above for wound care products based on an alginate wound care material, although the starting ingredients for the wound care material are different.

In the case of wound care products based on an amorphous hydrogel wound care material and comprising a polypeptide having wound care properties, these may be manufactured in a rather straightforward way that does not comprise fibre-spinning or drying: the polypeptide (optionally complexed to a bilayer-forming lipid) can simply be added during or after the gel-forming polymer and solvent are mixed to form the hydrogel.

In the case of wound care products based on a hydrogel sheet wound care material and comprising a polypeptide having wound care properties, these may also be manufactured in a rather straightforward way: the polypeptide (optionally complexed to a bilayer-forming lipid) can be added during or after the gel-forming polymer and solvent(s) are mixed but always before this mixture forms a hydrogel sheet by thermosetting, crosslinking or other process.

In the case of wound care products based on multiply layer dressings (such as those shown in FIG. 7), a film containing the polypeptide having wound care properties may be made separately and then applied to the wound care material layer. Alternatively, the film may be prepared on the wound care material layer by spray-coating, screen printing/roller-coat kissing, ultrasonic spraying and other techniques known in the art.

Finally, a fifth aspect of the invention provides a wound care kit comprising of a wound care material as defined above and an polypeptide having wound care properties as defined above.

Preferred aspects of the invention are described in the following non-limiting examples, with reference to the following figures:

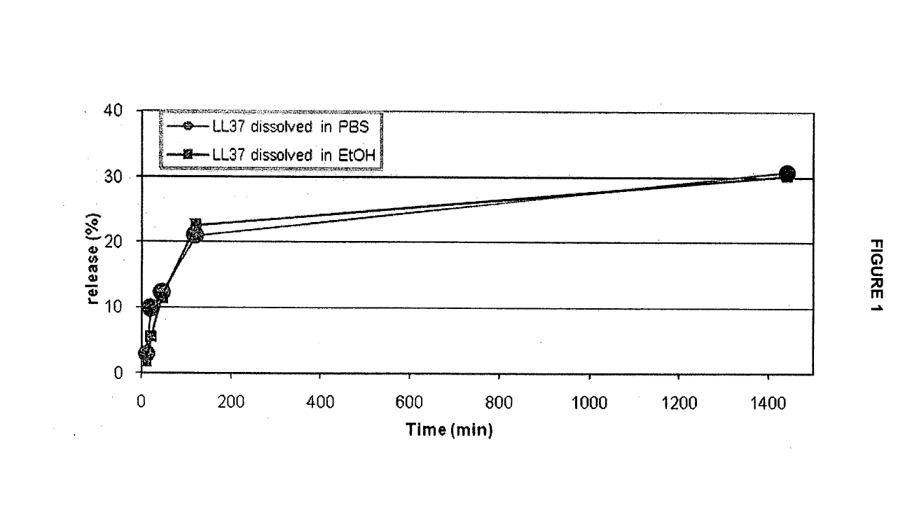

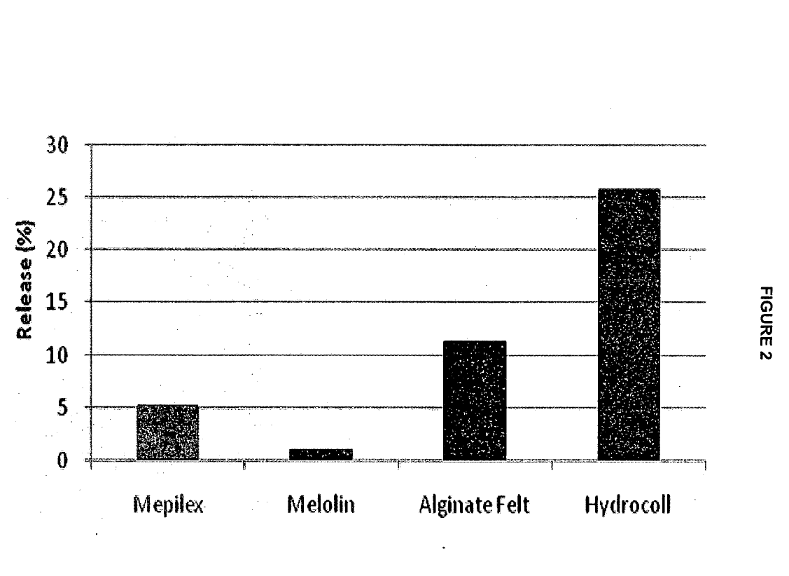

FIG. 1. Release of aqueous or ethanol solutions of LL-37 from PU foam.

LL-37 dissolved in PBS or ethanol was absorbed onto PU foam at a concentration of 25 μg LL-37/cm². A 1×1 cm piece of each preparation was cut, placed into a glass vial containing 3 ml PBS, and incubated under agitation for 24 h. At various time points (10, 20, 45, 120 min, and 24 h), 100 μl samples were collected and the amount of LL-37 released in solution was evaluated by ELISA. Results are expressed as the % LL-37 released in solution.

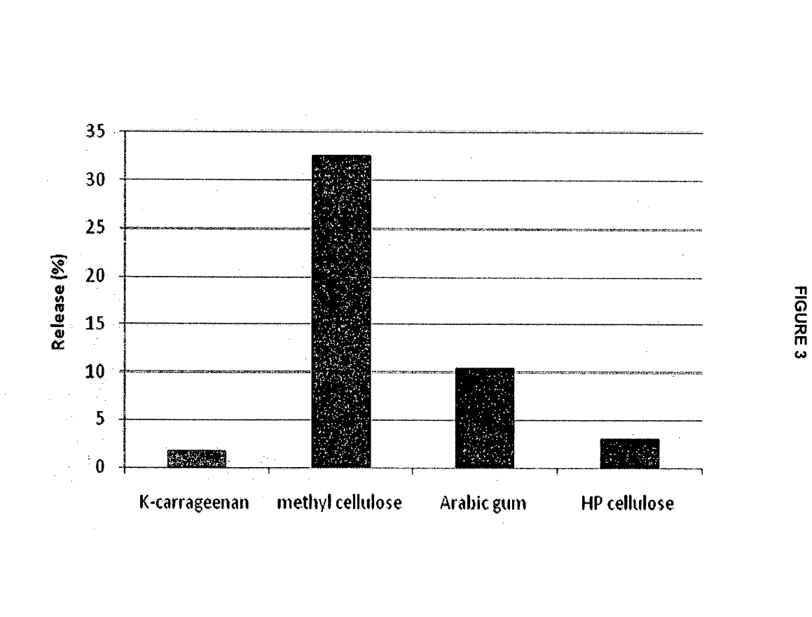

FIG. 2. Release of LL-37 from commercially available wound healing dressings.

LL-37 dissolved in PBS (250 μl at 100 μg/ml) was added on top of ˜1 cm² of different commercially available wound healing products. The materials were dried for and release of LL-37 was evaluated in 3 ml PBS-1% BSA after 24 h incubation. Results are expressed as the % LL-37 released in solution.

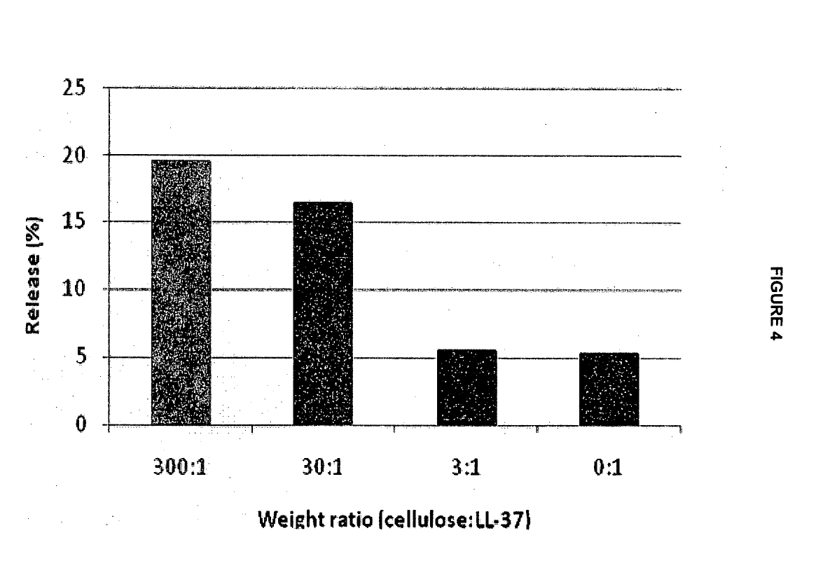

FIG. 3. Release of LL-37 in PBS-1% BSA from dried and rehydrated gels.

Aqueous solutions of LL-37 (100 μg/ml) were mixed with 5% K-carrageenan, 1% methyl cellulose, 5% Arabic gum, and 1.6% hydroxypropyl (HP) cellulose. Known amounts of gel were coated onto a glass surface and dried before being rehydrated with 3 ml PBS containing 1% BSA. The amount of LL-37 released from the gel was evaluated by ELISA and results expressed as % LL-37 released in solution being rehydrated with 3 ml PBS containing 1% BSA. The amount of LL-37 released from the gel was evaluated by ELISA and results expressed as % LL-37 released in solution.

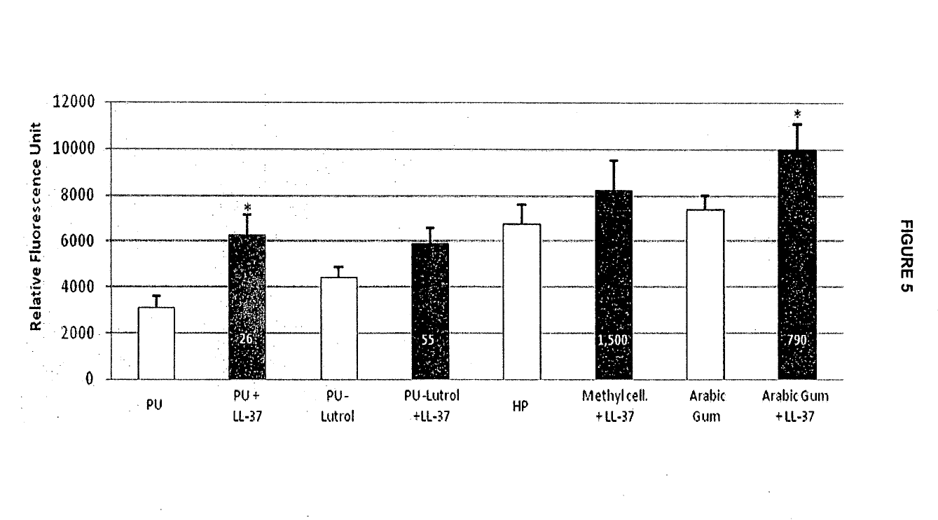

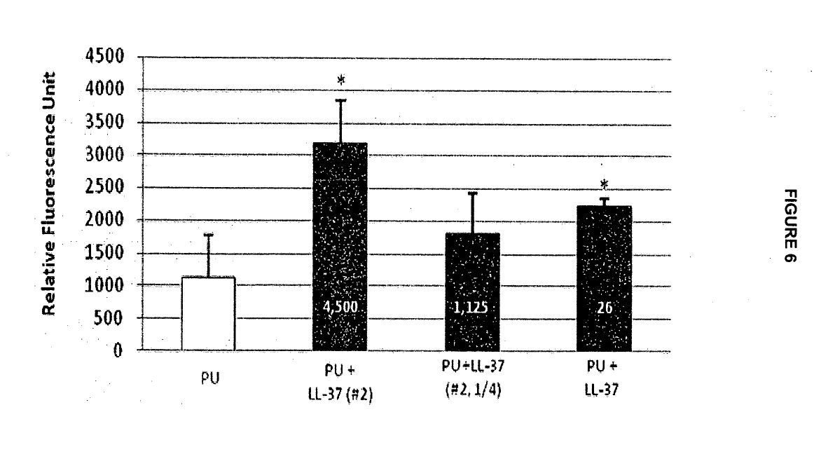

FIG. 4. Release of LL-37 from dried and rehydrated methyl cellulose gel composed of different gel:LL-37 ratios.

Aqueous solutions of LL-37 (100 μg/ml) were mixed with various amount of 1% methyl cellulose in order to get different weight:weight ratios (300:1, 30:1, and 3:1). As control, LL-37 was used in the absence of gel (0:1). Known amounts of gel were coated onto a glass surface and dried before being rehydrated with 3 ml PBS containing 1% BSA. The amount of LL-37 released from the gel was evaluated by ELISA and results expressed as % LL-37 released in solution being rehydrated with 3 ml PBS containing 1% BSA. The amount of LL-37 released from the gel was evaluated by ELISA and results expressed as % LL-37 released in solution.

FIG. 5. Chemotaxis of human PBMC toward various release samples.

Human PBMCs were isolated from fresh blood using Ficoll and resuspended in RPMI-1% BSA. Cells (5×10⁵ cells/ml) were allowed to migrate for 1.5 h toward 150 μl release sample in PBS-1% BSA. All migrated cells were then collected, DNA was stained with a fluorescent dye and fluorescence was evaluated. Each condition was measured in four replicates and the results are presented as average relative fluorescence unit (RFU) with the standard deviation. The control sample containing no LL-37 is represented with a white bar. Refer to Table 1 for the complete sample coding. * p<0.05 using 2-tail, unequal variance t-test when using the corresponding negative control. The number in the bars represent the LL-37 concentration (ng/ml) as determined by ELISA.

FIG. 6. Chemotaxis of human PBMC toward various release samples.

Human PBMCs were isolated from fresh blood using Ficoll and resuspended in RPMI-1% BSA. Chemotaxis assay was performed as described in FIG. 5. The control sample containing no LL-37 is represented with a white bar. Refer to Table 1 for the complete sample coding. * p<0.05 using 2-tail, unequal variance t-test when using the corresponding negative control. The number in the bars represent the LL-37 concentration (ng/ml) as determined by ELISA.

FIG. 7. Exemplary embodiments of wound care products of the invention
(A) Plan and side views of a simple dressing comprising a wound care material layer (such as PU foam) having attached on one side a water-soluble film containing LL-37. Note: Dressing not drawn to scale (e.g. film thickness is exaggerated).
(B) Plan and side views of a modified version of the simple dressing of (A) in which the film layer is perforated. Schematic diagram showing the initial absorption of wound exudate by wound care material through perforations in the film following later by release of LL-37 from the film.
   Note: Dressing not drawn to scale (e.g. perforation size is exaggerated).
(C) Plan and side views of a further modified version of the simple dressing of (A) in which the film layer is attached to the wound care material layer via an intervening film layer having slower dissolution rate than the film containing LL-37.

### EXAMPLE

### Formulation of LL-37, Evaluation of its Release from Various Devices, and Assessment of its Biological Activity

### Introduction

LL-37 is the only member of the human family of antimicrobial peptides called cathelicidins. LL-37 is derived from the human hCAP18 protein, expressed in various cell types and tissues (Durr, Sudheendra et al. 2006). Apart from exhibiting a broad antimicrobial spectra, it is now evident that LL-37 plays a broader role in host defense and also possesses wound healing properties (see Kai-Larsen and Agerberth 2008 and WO 2004/067025).

In one embodiment of the present invention, there is provided a class III medical device for use by people suffering from hard-to-heal or open wounds. The medical device may be composed of a dressing coated/impregnated/printed with a synthetically produced LL-37-containing formulation.

Material and Methods

Formulation of LL-37

Absorbing LL-37 into Polyurethane Foam

LL-37 was dissolved in ethanol or in PBS and 1 ml suspension was dropped onto 2×2 cm pieces of polyurethane (PU) foam (Brightwake, Kirkby-in-Ashfield, United Kingdom). The samples were allowed to dry at room temperature (RT) until no further weight loss could be recorded. Unless otherwise noted, a LL-37 solution at 100 μg/ml was used, resulting in a concentration of 25 μg LL-37/cm².

LL-37 was also mixed with various excipients before being applied onto the 2×2 cm PU foams. LL-37 dissolved in PBS was added to dry galactolipid and the resulting dispersion was vigorously shaken for 1 h. The galactolipid concentration was 0.2% (w/w). LL-37 dissolved in water was added to a preformed gel consisting of 25% poloxamer (Lutrol F127) in water. The resulting mixtures of about 1 g were heated at about 60° C. for about 10 min to evaporate the alcohol. The formulations were then applied with a spatula on 2×2 cm pieces of PU. In case the formulation contained water, PU foams were dried at RT for at least 24 h.

Applying/Absorbing LL-37 onto/into Commercially Available Wound Healing Products

LL-37 dissolved in PBS (250 μl at 100 μg/ml) was added on top of ˜1 cm² of different commercially available wound healing products: Duoderm (Convatec), Mepilex (Mölnlycke Health care), Melolin (Smith&Nephew), Alginate Felt and Hydrocoll (AG Hartmann) (only 50 μl of LL-37 solution was added). The materials were dried for 18 h at RT followed by 2 h at 37° C. LL-37 released from each samples was tested by adding 3 ml PBS or PBS containing 1% BSA for 24 h.

Evaporating a Mix of LL-37 Solution and a Gel Forming Excipient onto a Glass Surface

Aqueous solutions of LL-37 (100 μg/ml) were mixed with 1.6% hydroxypropyl (HP) cellulose (Apoteket, Stockholm, Sweden), 5% K-carrageenan (Sigma, Stockholm, Sweden), 1% methyl cellulose (Apoteket), 5% Arabic gum GO0020 (Scharlau). Known amounts of gel were coated onto a glass surface and dried at RT for 24 h followed by 3 h at 37° C. Gels were subsequently rehydrated and LL-37 release was studied after addition of 3 ml PBS or PBS containing 1% BSA to each vial.

Release of LL-37 from Various Devices

To release LL-37 from coated PU foam, 1×1 cm samples were cut and weighed in order to calculate the amount of LL-37 present in each sample. Each PU foam sample was placed into a 15 ml glass vial containing 3 ml PBS and incubated at RT for the indicated time period with constant shaking of 6 rpm. At various time intervals (10, 20, 45, 120 min, and 24 h), 100 μl release sample was removed for analysis and replaced with 100 μl fresh PBS. Release samples were stored at 4° C. until analysis, usually for 24 h.

To release LL-37 from glass coated with gel containing LL-37, 3 ml PBS was added to each vial, a sample was taken after 24 h and processed similarly as the above samples.

To release LL-37 from commercial wound healing products containing LL-37, 3 ml PBS was added to each vial, a sample was taken after 24 h and processed similarly as the above samples.

The release was calculated as the total amount of LL-37 in the release liquid divided by the amount of loaded LL-37 in the sample.

Detection and Quantification of Human LL-37 Released from Medical Devices

LL-37 was detected and quantified using an enzyme-linked immunosorbent assay (ELISA) based on the protocol developed by Lindgreen and colleagues (Lindgreen 2004). Medium binding capacity 96-well plates (Greiner Bio-one, Frickenhausen Germany) were coated with 5 μg/ml rabbit IgG anti-LL-37 antibodies (Agrisera, Vännäs, Sweden) in 200 μl coating buffer (0.1M bicarbonate buffer, pH 9.0) for 18 to 24 h at 2-8° C. Plates were washed 3 times with 200 μl washing buffer (0.01 M phosphate buffer pH 7.2, 0.145 M NaCl, and 0.2% Tween 20), blocked with 200 μl blocking solution (1% bovine serum albumin [BSA, Sigma] in 0.5 M Tris-HCl, pH 7.5) and washed as above. LL-37 standard (6.25-2,000 ng/ml) and samples (50 μl) were added to each well in duplicate followed by 150 μl dilution buffer (0.01 M PBS, 0.145 M NaCl, 0.1% Tween 20, and 0.1% BSA). Plates were incubated for 18 to 24 h at 2-8° C. After washing, 200 μl horseradish peroxidase (POD)-conjugated hen IgY anti-LL-37 (diluted 1/200 in dilution buffer) (Agrisera) were added. After 5 h incubation at room temperature with continuous shaking, samples were washed and developed for 30 min by adding 100 μl Color Reagent A and 100 μl Color Reagent B (TMB) (R&D Systems, Abingdon, United Kingdom). Reaction was stopped by addition of 50 μl 1 M sulfuric acid and absorbance immediately read at 450 nm (OD₄₅₀ₙₘ) using a VERSAmax microplate reader equipped with SoftMax Pro software for analysis (Molecular Devices).

Isolation of Human Peripheral Blood Mononuclear Cells

Peripheral blood mononuclear cells (PBMC) were isolated from venous blood (collected on K₂ EDTA) by Ficoll-Paque Plus (GE Healthcare, Uppsala, Sweden) centrifugation. Briefly, blood (30-50 ml) was diluted twice with room temperature (RT) Ca²⁺/Mg²⁺-free phosphate buffered saline (PBS, Invitrogen, Merelbeke, Belgium) and 30 ml of diluted blood was layered on top of 15 ml of Ficoll-Paque Plus. After 30 min centrifugation at 340 g and RT, the band corresponding to mononuclear cells was aspirated and the cells were washed two times with PBS before being resuspended in RPMI 1640 medium (Invitrogen) containing glutamax and supplemented with 100 μml penicillin (Invitrogen), 100 μg/ml streptomycin (Invitrogen), and 1% bovine serum albumin (BSA, Sigma, Stockholm, Sweden).

Chemotaxis Assay

Chemotaxis was assayed using the QCMT™ chemotaxis 96-well plates fitted with 3 μm membrane inserts (Millipore, Solna, Stockholm), according to the manufacturer's instructions. Briefly, 150 μl of chemoattractant test sample were distributed into the lower chamber of each well and 100 μl of cell suspension (5×10⁵ or 1×10⁶ cells/ml) were distributed into the upper chamber. Interleukin-8 (IL-8, R&D Systems) was used as positive control (10 and 100 ng/ml) and RPMI-1% BSA or PBS-1% BSA were used as negative controls to evaluate random migration. Chemoattractant samples were prepared either in RPMI-1% BSA or in PBS subsequently supplemented with 1% BSA. After 1.5 or 3 h incubation at 37° C. and 5% CO₂, migrated cells were recovered from the lower chamber and from the inserts according to the manufacturer's instructions. Cells were lysed and stained with a green fluorescent dye (CyQuant GR dye, Molecular Probes) for 15 minutes at room temperature. Cell lysate (150 μl) was transferred to a 96-well flat-bottomed opaque microplate (PerkinElmer, Upplands Väsby, Sweden) and fluorescence was read at 485/535 nm (1.0 s measurement time) using a Wallac 1420 fluorescent plate reader (Perkin Elmer). Each condition was performed in four replicates. Results are presented as mean relative fluorescence unit (RFU) or were converted to chemotactic index by dividing the average RFU of each sample by the average RFU of the appropriate negative control after subtracting the background RFU.

Reagents

The human cathelicidin antimicrobial peptide LL-37 (batches 990/37/A and 1013) (LLGDFFRKSKEKIGKEFKRIVQRIKDFLRNLVPRTES [SEQ ID NO:1]) was obtained from Polypeptide Laboratories A/S (Hillerod, Denmark). Unless otherwise noted, batch 1013 was used for all formulation experiments while batch 990/37/A was used for making standard curves for the ELISA. The peptide were reconstituted at 1 mg/ml in 1×PBS, aliquoted and stored at −20° C. until use.

Results

Formulation of LL-37

LL-37 was prepared in aqueous or non-aqueous solutions and either absorbed onto various supports, or mixed with gels.

When absorbing LL-37 solutions (PBS or ethanol) into PU foam, no change of the PU foam could be detected visually and all of the liquid used to dissolve LL-37 was easily removed after 2-3 days at room temperature.

LL-37 could be absorbed in all of the commercially available wound healing products with the exception of Duoderm and hydrocoll. For hydrocoll, smaller volumes of LL-37 solutions had to be used, which were successfully dried on top of the sample, leaving a salt-like spot on the membrane. The alginate sample became stiff after drying while the other two materials did not change shape.

All gels could be mixed with LL-37 solution without any precipitate formation (visual inspection).

LL-37 can be Released from Polyurethane Foam and Commercially Available Wound Healing Dressings

We first evaluated the release of LL-37 from PU foam after formulation of LL-37 in PBS, ethanol, or galactolipid. A 1×1 cm piece of each PU foam containing 25 μg LL-37 was incubated in 3 ml PBS under shaking conditions and samples were collected at various time points (10, 20, 45, 120 min, and 24 h) to measure the presence of LL-37 by ELISA. The results presented in FIG. 1 demonstrate that rapid release of LL-37 occurred from PU foam when the peptide was formulated in PBS or in ethanol. Release reached a maximum of 30% after 24 h incubation. No detectable or low (1%) release was observed when LL-37 was formulated in galactolipid (ethanol or PBS solutions) or in poloxamer (Lutrol) respectively (data not shown).

When applied to commercially available wound healing dressings, LL-37 formulated in PBS could also be released (FIG. 2). Best release was observed from the Hydrocoll product (25% release after 24 h) which contained 5 μg LL-37/cm² compared to 25 μg/cm² for the other dressings. The other dressings, Alginate Felt, Mepilex, and Melodin released about 10, 5, and 2% LL-37 respectively.

LL-37 can be Released from Dried and Rehydrated Gel-Forming Excipients

Aqueous solutions of LL-37 (100 μg/ml) were mixed with various gel forming products, coated onto a glass surface, dried, and subsequently rehydrated in 3 ml PBS for 24 h. The presence of LL-37 in each rehydrated gel was evaluated by ELISA and results are presented in FIG. 3. Various amount of LL-37 were released from the different gels, with methyl cellulose being the gel allowing the highest release of LL-37 (32%).

To evaluate if the amount of gel would influence the efficiency of LL-37 release, we performed an experiment in which various gel:LL-37 ratios were used to coat a glass vial (FIG. 4). In this case, we used methyl cellulose gel. As control, LL-37 alone (0:1) was dried onto a glass vial. After rehydrating the dried gels with 3 ml PBS containing 1% BSA, the presence of LL-37 was measured by ELISA. Surprisingly, the higher the amount of gel, the higher release of LL-37 was obtained (almost 20% release), suggesting that the addition of a gel to a LL-37 solution is not deleterious but actually favours its release from the device/support (here glass). Thus, addition of methyl cellulose increases the release of LL-37 from a solid support as compared to when LL-37 is added in PBS and dried before the release experiment.

The Released LL-37 Retains its Biological Activity

The functionality of LL-37 once released from various devices was evaluated using a chemotaxis assay which evaluates the ability of LL-37 to attract human cells. Chemotaxis is an important and relevant function to study in wound healing as recruitment of inflammatory cells occurs early during the normal wound healing process (Shai and Maibach 2005). In addition, LL-37 presents various biological activities, including chemotactic abilities (Kai-Larsen and Agerberth 2008).

Several release samples (Table 1) were evaluated for their ability to attract PBMCs (5×10⁵ cells/ml) after 1.5 h incubation.

TABLE 1
List of samples evaluated for their chemotactic ability

| Name | Description* |
|---|---|
| PU | Polyurethane (PU) foam |
| PU + LL-37 (#2) | PU foam coated with 32 μg/cm² LL-37 dissolved in PBS, sample #2 |
| PU + LL-37 (#2, 1:4) | PU foam coated with 32 μg/cm² LL-37 dissolved in PBS, sample #2, |
| | diluted ¼ |
| PU + LL-37 | PU foam coated with 12 μg/cm² LL-37 dissolved in PBS, sample #3 |
| PU-lutrol | PU foam coated PBS dissolved in poloxamer (lutrol L127) |
| PU-lutrol + LL-37 | PU foam coated with 25 μg/cm² LL-37 dissolved in poloxamer (lutrol L127) |
| HP | Hydroxypropyl (HP) cellulose gel (4 mg/ml in PBS) |
| Methyl cell. + LL-37 | Methyl cellulose gel containing 25 μg LL-37 (metyl:LL-37 = 300:1) |
| Arabic Gum | Arabic Gum gel |
| Arabic Gu + LL-37 | Arabic Gum gel containing 25 μg LL-37 (gum:LL-37 = 1,500:1) |

*See Material and methods section “Formulation of LL-37” for a complete description of the samples.

The results presented in FIG. 5 demonstrate that samples released from PU foam coated with LL-37 in PBS or from Arabic gum mixed with LL-37 significantly attract more PBMCs compared to the samples released from untreated PU on unmodified Arabic gum respectively. There was a trend for increased chemotaxis activity of PU foam coated with LL-37-containing poloxamer (Lutrol); however, the increase was however not significant (p=0.054). This result indicates that LL-37-coated foam or formulated into a gel can release LL-37 in suspension and that the released peptide retains its biological activity.

To confirm these results and rule out donor-specific results, a second experiment was carried out, using blood from a different donor. In this case, release from two PU foams where LL-37 in PBS has been absorbed (25 μg LL-37/cm²) was evaluated (FIG. 6). Because one samples one known to contain high amount of LL-37 (4,500 ng/ml), dilution (1/4) of that sample was also evaluated for its chemotactic ability.

### CONCLUSIONS

The results demonstrate that biologically active LL-37 can be released from a preloaded and dried dressing upon contact with a water-containing solution. Furthermore, the release of bioactive LL-37 from the dressing enhances the function of leukocytes, as exemplified here by active migration through 3 μm size pores.

### REFERENCES

Carretero, M., M. J. Escamez, et al. (2008). “In vitro and in vivo wound healing-promoting activities of human cathelicidin LL-37.” J Invest Dermatol 128(1): 223-36.
De, Y., Q. Chen, et al. (2000). “LL-37, the neutrophil granule- and epithelial cell-derived cathelicidin, utilizes formyl peptide receptor-like 1 (FPRL1) as a receptor to chemoattract human peripheral blood neutrophils, monocytes, and T cells.” J Exp Med 192(7): 1069-74.
Durr, U. H., U. S. Sudheendra, et al. (2006). “LL-37, the only human member of the cathelicidin family of antimicrobial peptides.” Biochim Biophys Acta 1758(9): 1408-25.
Heilborn, J. D., M. F. Nilsson, et al. (2003). “The cathelicidin anti-microbial peptide LL-37 is involved in re-epithelialization of human skin wounds and is lacking in chronic ulcer epithelium.” J Invest Dermatol 120(3): 379-89.
Kai-Larsen, Y. and B. Agerberth (2008). “The role of the multifunctional peptide LL-37 in host defense.” Front Biosci 13: 3760-7.
Kaplan, S. S., R. E. Basford, et al. (1990). “Biomaterial associated impairment of local neutrophil function.” ASAIO Trans 36(3): M172-5.
Lindgreen, P. (2004). “Development of LL37 ELISA for LL37 Quantification in Human and Minipig Serum (R8).” 76.
Nagaoka, I., H. Tamura, et al. (2006). “An antimicrobial cathelicidin peptide, human CAP18/LL-37, suppresses neutrophil apoptosis via the activation of formyl-peptide receptor-like 1 and P2X7.” J Immunol 176(5): 3044-52.
Shai, A. and H. I. Maibach (2005). Wound healing and ulcers of the skin, Springer.
Shaykhiev, R., C. Beisswenger, et al. (2005). “Human endogenous antibiotic LL-37 stimulates airway epithelial cell proliferation and wound closure.” Am J Physiol Lung Cell Mol Physiol 289(5): L842-8.
Sieber, V. K., W. R. Otto, et al. (1995). “Cytotoxicity of wound dressing materials assessed using cultured skin equivalents.” Burns 21(4): 249-54.
Tokumaru, S., K. Sayama, et al. (2005). “Induction of keratinocyte migration via transactivation of the epidermal growth factor receptor by the antimicrobial peptide LL-37.” J Immunol 175(7): 4662-8.

1-92. (canceled)

93. A wound care product comprising a wound care material and a polypeptide having wound healing properties
wherein the polypeptide having wound healing properties is a cathelicidin, or a fragment, variant or fusion thereof which retains, at least in part, the wound healing activity of said cathelicidin.

94. A wound care product according to claim 93 wherein the cathelicidin is selected from the group consisting of human cationic antimicrobial protein (hCAP18), PR39, prophenin and indolicidin.

95. A wound care product according to claim 93 wherein the polypeptide having wound healing properties is LL-37.

96. A wound care product according to claim 93 wherein the wound care material is selected from the group consisting of alginates, amorphous hydrogels, sheet hydrogels, hydrofibres, foams, hydrocolloids, collagen-based materials, hyaluronic acid based materials, dextranomers, dextrinomer/cadexomer and oxidised regenerated cellulose and mixtures thereof.

97. A wound care product according to claim 93 wherein the wound care material comprises or consists of an amorphous hydrogel.

98. A wound care product according to claim 97 wherein the hydrogel comprises one or more hydrogel-forming polymers selected from the group consisting of synthetic polymers, such as polyvinylalcohol, polyvinylpyrolidone, polyacrylic acid, polyethylene glycol, poloxamer block copolymers and the like; semi-synthetic polymers, such as cellulose ethers, including carboxymethylcellulose, hydroxyl-ethylcellulose, hydroxypropylcellulose, methylcellulose, methyl-hydroxypropylcellulose and ethylhydroxyethylcellulose, and the like; natural gums, such as acacia, carragenan, chitosan, pectin, starch, xanthan gum and the like; and alginates.

99. A wound care product according to claim 93 wherein the wound care material comprises or consists of a sheet hydrogel.

100. A wound care product according to claim 99 wherein the hydrogel comprises one or more hydrogel-forming polymers selected from the group consisting of synthetic polymers, such as polyurethanes, polyvinylalcohol, polyvinylpyrolidone, polyacrylic acid, polyethylene glycol, poloxamer block copolymers and the like; semi-synthetic polymers, such as cellulose ethers, including carboxymethylcellulose, hydroxyethylcellulose, hydroxypropyl-cellulose, methylcellulose, methylhydroxypropylcellulose and ethylhydroxyethylcellulose, and the like; natural gums, such as acacia, carragenan, chitosan, pectin, starch, xanthan gum and the like; and alginates.

101. A wound care product according to claim 93 wherein the wound care material comprises or consists of polyurethane foam and the polypeptide having wound healing properties is LL-37.

102. A wound care product according to claim 93 wherein the polypeptide having wound healing properties is capable of being released slowly in use.

103. A wound care product according to claim 93 wherein the wound care product is for maintaining a moist wound environment.

104. A wound care product according to claim 93 wherein the wound care product is capable of preventing, abolishing, reducing or otherwise diminishing microbial growth in a wound environment.

105. A wound care product according to claim 93 wherein the wound care product is capable of enhancing epithelial regeneration and/or healing of wound epithelia and/or wound stroma.

106. A wound care product according to claim 93 comprising a layer of wound care material to which is attached on the wound-facing side a film containing the polypeptide having wound healing properties.

107. A wound care product according to claim 106 wherein the wound care material layer comprises or consists of a polyurethane foam dressing, a hydrocolloid sheet dressing, a hydrogel sheet or a non-aqueous gel sheet.

108. A wound care product according to claim 106 wherein the film is equal to or less than 1 mm thick.

109. A wound care product according to claim 106 wherein the film is perforated.

110. A wound care product according to claim 106 selected from the following:
a. A wound care product capable of absorbing wound exudate comprising a polyurethane foam dressing to which is attached, on the side to be contacted with the wound, a non-perforated water-soluble film containing LL-37;
b. A wound care product capable of absorbing wound exudate comprising a polyurethane foam dressing to which is attached, on the side to be contacted with the wound, a perforated water-soluble film containing LL-37;
c. The wound care product of (a) or (b) wherein the film is attached indirectly to the polyurethane foam dressing via a non-perforated water-soluble intervening layer (having lower water-solubility than the film); and
d. The wound care product of (a) or (b) wherein the film is attached indirectly to the polyurethane foam dressing via a perforated water-soluble intervening layer (having lower water-solubility than the film).

111. A method for treating a wound comprising contacting the wound with wound care product according to claim 93.

112. A method for producing a product according to claim 93 comprising combining a wound care material and a polypeptide having wound healing properties.
